(19) United States
(12) Patent Application Publication
(10) Pub. No.: US 2014/0127687 A1
(43) Pub. Date: May 8, 2014

(54) Method for Separation, Determination or Enrichment of Different DNA Species

(71) Applicant: Analytik Jena AG, Jena (DE)

(72) Inventors: Dirk Osterloh, Jena (DE); Karen Felsmann, Jena (DE)

(73) Assignee: Analytik Jena AG, Jena (DE)

(21) Appl. No.: 14/056,756

(22) Filed: Oct. 17, 2013

(30) Foreign Application Priority Data
Oct. 19, 2012 (DE) 102012219142.9

(51) Int. Cl. C12Q 1/68

(52) U.S. Cl. CPC C12Q 1/689
USPC 435/6.11

(57) ABSTRACT

The present invention relates to a method for separating, determining or accumulating different DNA species that are present simultaneously in a sample, on the basis of unmethylated CpG-dinucleotides occurring with different frequency, within each DNA species present, wherein an unmethylated CpG motifs binding protein is immobilized on a base material and the DNA bound to the immobilized protein is eluted using an elution agent gradient, wherein specific concentration regions of the elution agent used correlate with specific CpG-dinucleotide frequencies in the DNA species present, so that during elution different fractions of DNA species are obtained that have different frequencies of CpG-dinucleotides.

### PRIORITY CLAIM

This application is a United States Non-provisional application claiming priority under 35 U.S.C. §119 from German Patent Application No. DE 102012219142.9, filed Oct. 19, 2012, the entire contents of which are herein incorporated by reference.

### DESCRIPTION

The present invention relates to an in vitro method for separation, determination or enrichment of different DNA species that are simultaneously present in a sample, on the basis of unmethylated CpG-dinucleotides occurring with varying frequency within each DNA species present, unmethylated CpG-dinucleotides being those that do not comprise a methyl group in position 5 of the cytosine, wherein

the DNA that contains several different DNA species, is isolated from the sample and an aqueous solution of the isolated DNA is produced;

the DNA solution is brought into contact with at least one protein being immobilized on a base material and binding unmethylated CpG-dinucleotides;

unspecifically bound DNA and methylated DNA is eluted; and

the base material is eluted using at least one elution agent that has different concentrations, specific concentration regions of the elution agent used correlating with specific CpG-dinucleotide frequencies in the DNA species present, so that during elution different fractions of DNA species are obtained that have different frequencies of CpG-dinucleotides.

Nucleic acid amplification techniques (NAT such as, for example, polymerase chain reaction, PCR) allow for the highly sensitive and highly specific determination of microbial nucleic acids and thus could be an excellent tool for improving the determination and identification of bacteria, fungi and viruses. In both human and veterinary medicine there is the problem in clinical samples that the microorganisms to be determined for diagnostic purposes usually only occur in very small number and thus only few target DNA molecules are present within the samples.

The situation is rendered more difficult by the fact that typical clinical samples such as tissue or blood samples include a high DNA background caused by host cells, an excess of the host DNA typically existing that comprises plural decimal powers.

For efficient microbial determination systems that are based on nucleic acid amplification techniques, effective and standardized methods are therefore required that are performed prior to an actual analysis and/or determination (pre-analytic procedures).

Effective pre-analytic procedures have to cope with eliminating a large number of inhibiting and interfering chemical compounds that in general are present in biological samples such as, e.g. salts, proteins, lipids, metabolites of pharmaceuticals and competitive nucleic acids. In the prior art, there are a number of preparation techniques that allow for separating salts, proteins, lipids, organic compounds and RNA. Those techniques inter alia include precipitation, protease or ribonucleic acid digestion, liquid and solid-phase extraction. However, up to the present date no effective separation of relevant bacterial or fungal DNAs from interfering eukaryotic DNAs is available.

Although it is basically possible to separate prokaryotic and eukaryotic DNAs on the basis of their typical differences in molecular weight, melting properties and epigenetic properties, this requires extensive equipment and moreover takes a long time and requires sufficient quantities of eukaryotic DNA.

From an analytic point of view, however, such a separation of eukaryotic and prokaryotic DNA would be desirable since this would make it possible to more securely and sensitively determine DNA of micro-organisms.

In order to approach the goal of separating prokaryotic and eukaryotic DNA, different methylation patterns may be helpful that are typical of prokaryotic and eukaryotic DNA, in particular of genomic DNA of mammals.

In this connection it is especially known that 5-methylcytosine, a so-called epigenetic modification or posttranslational modification of the nucleobase cytosine, merely occurs in genomic DNA of eukaryotes, but virtually not in prokaryotes.

DNA methylation in eukaryotes is based on the substitution of an H atom for a methyl group in the 5 position of a cytosine pyrimidine ring.

It is known that DNA methylation plays a central role in the normal development of organisms and in cellular differentiation in superior organisms. DNA methylation stably changes the gene expression pattern of cells or lowers gene expression and provides the basis for forming a chromatin structure. From the prior art it likewise is known that DNA methylation plays a central role in the development of neoplasiae (Jaenisch and Bird, 2003: Epigenetic regulation of gene expression: how the genome integrates intrinsic and environmental signals; Nature Genetics 33 Suppl: 245-254).

### DNA Methylation in Mammals

The substitution of a hydrogen atom in the 5-position of a cytosine ring for a methyl group in CpG dinucleotides constitutes the main epigenetic change in the genome of a mammal. This was confirmed as yet for each examined member of vertebrates.

In particular, between 60% and 90% of all CpG nucleotides in mammals are methylated (Ehrlich et al, 1982 Amount and distribution of 5-methylcytosine in human DNA from different types of tissues of cells. Nucleic Acids Research 10 (8): 2709-2721 and Tucker K L, 2001, Methylated cytosine and the brain: a new base for neuroscience. Neuron 30 (3): 649-652). On account of their chemical structure methylated cytosine nucleobases may deaminate spontaneously in order to form thymine nucleobases. As a consequence, methylated CpG dinucleotides regularly mutate to form TpG dinucleotides. A hint to the presence of such mutations is an under-representation of CpG dinucleotides in the human genome as they merely occur therein by approximately 21% of the expected frequency (International Human Genome Sequencing Consortium, 2001 “Initial sequencing and analysis of the human genome. Nature 409 (6822): 860-921”). On the other hand, spontaneous deamination of unmethylated cytosine nucleobases leads to the conversion of same to uracil, a mutation which, however, is quickly recognized and repaired by the cell.

Unmethylated CpGs frequently are grouped in so-called clusters that are referred to as CpG islands. They lie in the 5′-regulatory areas of many genes. In numerous disease processes such as cancer, for example, the CpG islands in gene promoters exhibit abnormal hypermethylation patterns that lead to transcriptional silencing, which subsequently to the cell division can be inherited to the daughter cell.

### DNA Methylation in Fungi

Typically, fungi exhibit very low levels of cytosine methylation. The degree of methylation varies between 0.1 and 5%, depending on the species (Antequera et al. 1984 DNA methylation in the fungi. J. Biol. Chem. 259 (13): 8033-8036).

In particular, the above-indicated methylation values also appear to vary within the same species (confer Binz et al., 1998 “A comparison of DNA methylation levels in selected isolates of higher fungi. Mycologia (Mycological Society of America) 90 (5): 785-790”).

### DNA Methylation in Bacteria

While eukaryontic DNA methylation exclusively relates to cytosine residues within the DNA and is specific for CpG motifs both adenine and cytosine residues may be methylated in bacteria.

So far, a number of DNA methyltransferases (DNA-MTase) has been discovered that catalyze cytosine methylation in the different sequence context (Noyer-Weidner and Trautner, 1993 “Methylation of DNA in prokaryotes. EXS 64, 39-108”). Thus, most strains of E. coli, for example, include sequence-specific DNA methylases:

Dam methylase: Methylation on the N⁶-position of adenine in the sequence GATC

Dcm methylase: Methylation on the C⁵-position of cytosine in the sequence CCAGG and CCTGG

EcoKi methylase: Methylation of adenine in the sequences AAC (N⁶A) GTGC and GCAC (N⁶A) GTT.

The main function of DNA methylation in bacteria is to provide a mechanism in order to protect the bacteria cells against self-digestion as well as against the intrusion of foreign DNA. Bacterial restriction endonucleases may differentiate between endogenous DNA and foreign DNA by way of their methylation pattern. For example, phages-introduced DNA which is not protected by methylation corresponding to the host cell, is eliminated by cleavage (Noyer-Weidner and Trautner, 1993 “Methylation of DNA in prokaryotes. EXS 64, 39-108”).

It is known in the prior art that the GC content in genomes of different species, particularly of bacterial species, varies strongly. Within the domain of bacteria GC contents of between 25 and 75% were measured (confer Hill, L. R. 1966. An Index to deoxyribonucleic acid base compositions of bacterial species. J. Gen. Microbiol. 44:419-437). On account of the different GC contents of species within one genus and the phylogenetic differences proposals were made in the prior art to classify bacteria by way of their GC content (confer Wayne et al, 1987 “Report of the ad hoc committee on reconciliation of approaches to bacterial systematic. International journal of systematic bacteriology 37 (4): 463-4”).

A large diversity of GC contents with values of 26-70% was also determined in fungi (confer Storck, R and C. J. Alexopoulos, 1970. Deoxyribonucleic acid of fungi. Bacteriol. Rev. 34(2): 126), in protozoa with values of 22-68%, and in algae with values of 37-68% (confer Mandel, M. 1967 Nucleic acids of protozoa, p. 541-572. In Florkin, M., Scheer, B, T, and G. W. Kidder, Chemical zoology, vol. I Academic Press Inc., New York) as well as in cyanobacteria with values of 35-71% (confer Edleman, M., Swinton, D., Schiff, J. A., Epstein, H. T., and B. Zeldin, 1967. Deoxyribonucleic acid of the blue-green algae (cyanophyta). Bacteriol. Rev. 31:315-331).

In the following, the GC contents of selected model organisms are rendered in table 1.

TABLE 1

| | phylogenetic | |
|---|---|---|
| Species | Classification | GC-content* |
| Streptomyces | Actinobacterium | 72% |
| coelicolor | | |
| Myxococcus xanthus | Deltaproteobacterium | 68% |
| Halobacterium sp. | Archaeon | 67% |
| Saccharomyces | Ascomycete (fungus) | 38% |
| cerevisiae | | |
| Arabidopsis thaliana | Flowering plant | 36% |
| (Thale cress) | | |
| Methanosphaera | Archaeon | 27% |
| stadtmanae | | |
| Plasmodium | Protozoon | ~20% |
| falciparum | | |

*In general, the GC content is expressed in percentage. The percentage of GC content is calculated as [G + C/A + T + G + C] × 100.

Actinobacteria, for example, is characterized in the taxonomy browser of the National Center for Biotechnology Information [NCBI]. According to the overall genome data of the NCBI, the GC content in streptomyces coelicolor A3 (2) is 72%. According to the overall genome data of the NCBI, the GC content of yeast (Saccharomyces cerevisiae) is 38% and the GC content of a further model organism frequently used, i.e. thale cress (Arabidopsis thaliana) is 36%.

It is well known to a skilled person that on account of the nature of the genetic code it is virtually impossible for an organism to have a genome with a GC content of either 0% or 100%. However, a species of the malaria pathogen Plasmodium falciparum with an extremely low GC content (GC≈20%) according to the overall genome data of Plasmodium falciparum of the NCBI is known and it has become a habit in the scientific community to rather consider those organisms as being “AT-rich” than refer to them as being “GC-poor” (confer Musto et al., 1997 “Compositional constraints in the extremely GC-poor genome of Plasmodium falciparum. Mem. Inst. Oswaldo Cruz 92 (6): 835-51”).

It is important to know that the GC-proportions within a genome may vary considerably. Thus, the GC-content of human DNA, for example, strongly varies over the genome, starting with 30% to 60%, and these variations in the GC-proportion within the genome of more complex organisms therefore lead to a mosaic-like formation with island regions, i.e. so-called isochores (confer Bernardi, 2000 “Isochores and the evolutionary genomics of vertebrates”. Gene 241 (1): 3-17)”).

GC-rich isochores include many protein-encoding genes and therefore a determination of the proportions in those specific regions may contribute to mapping gene-rich regions of the genome (confer Sumner et al., 1993 “The distribution of genes on chromosomes: a cytological approach.” J. Mol. Evol. 37 (2): 117-22; Aissani and Bernardi, 1991 “CpG islands, genes and isochores in the genome of vertebrates.” Gene 106 (2): 185-95).

As was already mentioned at the beginning, such GC-rich regions and particularly CpG islands may be methylated or unmethylated on the cytosine.

Basically, it is to be ascertained that bacteria include unmethylated CpG motifs, whereas in particular human DNA includes methylated CpG motifs.

From the prior art according to EP 1 400 589 B1 as well as WO 2004/033683 A1 it is known to use unmethylated CpG-DNA binding proteins for enrichment and/or separation of bacterial DNA of human DNA in samples in which a strong excess of human DNA is present.

Selective binding of specific proteins to unmethylated CpG-DNA is procured by the CXXC protein domain (zf-CXXC; Pfam PF02008). So far, a number of proteins that include the CXXC domain were described in the prior art. Those are e.g. the protein which binds to the methyl-CpG binding domain (MBD1) (confer Cross et al., 1997 “A component of the transcriptional repressor MeCP1 shares a motif with DNA methyltransferase and HRX proteins. Nat Genet 16: 256-259”), DNA methyltransferase 1 (DNMT1) (confer Bestor and Verdine, 1994 “DNA methyltransferases. Curr Opin Cell Biol 6: 380-389”), the main DNA repair DNA methyltransferase (FBXL11) that recently was characterized as histone demethylase which specifically demethylates histone H3 amino lysine 36 (Tsukada et al., 2006 “Histone demethylation by a family of JmjC domain-containing proteins. Nature 439: 811-816”), as well as the CpG binding protein (CGBP), a component of the mammalian Set1 H3-H4 methyltransferase complex (Lee and Skalnik, 2005 “CpG-binding protein (CXXC finger protein 1) is a component of the mammalian Set1 histone H3-Lys4 methyltransferase complex, the analogue of the yeast Set1/COMPASS complex. J Biol Chem 280: 41725-41731”).

The cysteine-rich CXXC domain is found in a number of chromatin-associated proteins and is responsible for the protein specifically binding unmethylated CpG-dinucleotides. The CXXC domain has two CGXCXXC repeats. As far as is known, the CXXC domain contains eight preserved cysteine residues, without being bound thereto, which cysteine residues are apt to bind two zinc ions. The molecular basis of identifying unmethylated CpG DNA was examined especially by Allen et al., 2006 “Solution structure of the nonmethyl-CpG-binding CXXC domain of the leukaemia-associated MLL histone methyltransferase; The EMBO Journal 25, 4503-4512”, who determined the spatial structure of the CXXC domain of the human CXXC protein MLL by way of multi-dimensional NMR spectroscopy. According to their findings, the CXXC domain has a fold in which two zinc ions are coordinated in the shape of a tetrahedron by four preserved cysteine ligands that are provided by two CGXCXXC motifs and two distal cysteine residues. Allen et al., 2006 also measured the binding affinity of the CXXC domain vis-à-vis DNA molecules that comprise a central unmethylated CpG-dinucleotide. They found that the CXXC domain binds a 12-mer DNA having a central CpG-motif with a binding affinity of K_{d} of 4.3 μM±0.4 μM. No binding was observed for the same DNA molecule in case it included a central methylated CpG-motif.

Based on the prior art according to EP 1 400 589 B1 it is therefore an object of the present invention to provide a method for separating, determining or enriching different DNA species by which e.g. a simple and reliable taxonomical determination of bacterial and fungal microorganisms is made possible.

In particular, the present invention relates to an in vitro method for separating, determining or enriching different DNA species that are present simultaneously in a sample, on the basis of unmethylated CpG-dinucleotides occurring with varying frequency, within each DNA species present, unmethylated CpG-dinucleotides being those that do not comprise a methyl group in position 5 of the cytosine, wherein the DNA that contains several different DNA species, is isolated from the sample and an aqueous solution of the isolated DNA is produced;

the DNA solution is brought into contact with at least one protein being immobilized on a base material and binding unmethylated CpG-dinucleotides;

unspecifically bound DNA and methylated DNA is washed out; and

the base material is eluted using at least one elution agent that has different concentrations, specific concentration regions of the elution agent used correlating with specific CpG-dinucleotide frequencies in the DNA species present, so that during elution different fractions of DNA species are obtained that have different frequencies of CpG-dinucleotides.

Such different DNA species may originate from different bacteria and/or fungi.

Thus, in accordance with the present invention, DNA species from different bacteria and fungi may be separated.

For the purpose of the present invention the term CpG-dinucleotide is to mean a two nucleobases long region within a DNA that includes the components of cytosine-phosphate-guanine in the reading direction of a 5′ end to a 3′ end and that can be rendered, for example, by the following structure:

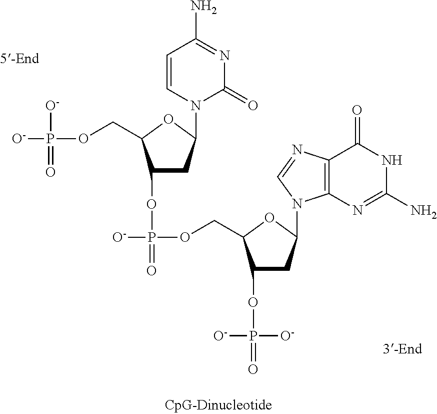 [SMILES: Nc1ccn([C@H]2C[C@@H](OP(=O)([O-])OC[C@H]3O[C@@H](n4cnc5c(=O)[nH]c(N)nc54)C[C@H]3OP(=O)([O-])[O-])[C@@H](COP(=O)([O-])[O-])O2)c(=O)n1]

By definition, unmethylated CpG dinucleotides are those that do not comprise a methyl group in position 5 of the cytsine, i.e. do not include a nucleobase residue in accordance with the following chemical structure:

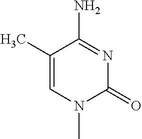

Such CpG-dinucleotides also are referred to as CpG-motifs.

A preferred method in accordance with the invention is characterized in that the different DNA species originate from taxonomically different microorganisms. These microorganisms preferably are bacteria and/or fungi that are selected in particular from the group consisting of:

the genera of bacteria:

Pseudomonas; Stenotrophomonas; Neisseriaceae; Echerichia; Proteus; Acinetobacter; Streptococcus; Staphylococcus; Costridium;

the species of bacteria:

Pseudomonas aeroginosa, Stenotrophomonas maltophila, Neisseria meningitidis, Echerichia coli, Proteus mirabilis, Acinetobacter baumannii, Streptococcus pneumoniae, Staphylococcus aureus, Clostridium perfringens;

the fungus genera of Aspergillus and Saccharomyces; and

the fungus species of Aspergillus niger and Saccaromyces cerevisiae.

In a preferred alternative of the method in accordance with the invention the determination of different DNA species is used for identifying microorganisms, in particular genera of bacteria and/or fungi and/or protozoa, and/or algae, and/or cyanobacteria and/or species of bacteria and/or fungi, and/or species of protozoa and/or algae and/or cyanobacteria.

A likewise preferred embodiment of the present method in accordance with the invention consists in that the identification of microorganisms/bacteria is done on the basis of the detected DNA species in the sample via the following association of frequencies of the CpG-dinucleotide within the detected DNA species, expressed as reciprocal of the frequency (ν) of CpG-dinucleotides, the frequency being the number of nucleotides [nt] in a DNA species that statistically accounts for one CpG-dinucleotide:

| Species of microorganism | 1/ν CpG-dinucleotide [nt] |
|---|---|
| Pseudomonas aeruginosa | 36.0-36.4, preferably 36.2 |
| Stenotrophomonas maltophilia | 36.5-36.8, preferably 36.6 |
| Neisseria meningitidis | 42.4-43.2, preferably 42.8 |
| Echerichia coli | 55.1-55.9, preferably 55.5 |
| Proteus mirabilis | 111.5-112.6, preferably 112.1 |
| Acinetobacter baumannii | 120.9-121.9, preferably 121.4 |
| Streptococcus pneumoniae | 144.5-145.5, preferably 145.0 |
| Staphylococcus aureus | 159.2-160.2, preferably 159.7 |
| Clostridium perfringens | 801.7-805.7, preferably 803.7 |

Advantageously, in accordance with the present invention, the content of the nucleobases guanine and cytosine (GC-content) may be used as measurement of the frequency of a CpG-dinucleotide in a DNA species, expressed in % as

$$[ \frac{mGuanine + mCytosine}{mAdenine + mThymine + mGuanine + mCytosine} ] \times 100$$

m referring to the mass of the respective nucleobase.

A further preferred embodiment of the method in accordance with the invention is characterized in that the base material is eluted using a salt solution, in particular a salt gradient, preferably an (NH₄)₂CO₃-gradient or an NaCl gradient having an NaCl concentration of 0.01 M to 1.0 M, particularly 0.1 M to 0.8 M, preferably 0.2 M to 0.7 M, wherein specific concentration regions correspond to a specific CpG frequency and/or a specific GC-content, so that individual DNA fractions are obtained that have different CpG frequencies and/or different GC-contents; and

comparing the salt concentrations of the individual eluted DNA fractions with the salt concentration of eluted DNA with a known CpG frequency and/or known GC-content of known microorganisms in order to identify an unknown microorganism on the basis of its CpG frequency and/or GC-content in the respective DNA fraction.

Preferably, the salt concentration of the individual DNA fractions is determined via a measurement of the electrical conductivity. This constitutes a simple measurement that can be performed quickly, for which a number of measuring instruments are available in the prior art.

A further preferred embodiment of the method in accordance with the invention is characterized in that the unmethylated CpG-dinucleotides binding protein is selected from the group consisting of: proteins containing at least one CXXC domain; protein fragments comprising at least one CXXC domain, particularly human non-methyl-CpG binding protein CXXC1/CGBP as well as its isoforms 1 and 2 and fragments thereof that include the CXXC domain; fusion proteins including the CXXC domain, in particular fusion proteins that include sequences of the human non-methyl-CpG binding protein CXXC1/CGBP.

In particular, the CXXC domain includes eight preserved cysteine residues that are able to bind two zinc ions. The domain is characterized by two CGXCXXC sequence motifs. The CXXC domain binds unmethylated CpG dinucleotides.

Confer Cross S H, Meehan R R, Nan X, Bird, A; Nat Genet 1997; 16: 256-259: A component of the transcriptional repressor MeCP1 shares a motif with DNA methyltransferase and HRX proteins; Bestor T H; EMBO J 1992; 11:2611-2617: Activation of mammalian DNA methyltransferase by cleavage of a Zn binding regulatory domain; as well as Allen M D, Grummitt C G, Hilcenko C, Min S Y, Tonkin L M, Johnson C M, Freund S M, Bycroft M, Warren A J; EMBO J. 2006; 25:4503-4512: Solution structure of the nonmethyl-CpG-binding CXXC domain of the leukaemia-associated MLL histone methyltransferase; and Chao Xu, Chuanbing Bian, Robert Lam, Alping Dong & Jinrong Min; Nat. Commun. 2011; 2:227: The structural basis for selective binding of non-methylated CpG islands by the CFP1 CXXC domain.

Moreover, it is preferred that the unmethylated CpG-dinucleotides binding protein on its N-terminus or its C-terminius is fused with an amino acid sequence that has an affinity for proteins and peptides, for example, antibodies (c-myc-tag, FLAG-tag), for streptavidin (Strep-tag), for calmodulin (calmodulin-binding oligopeptide), or glutathione (glutathione-S-transferase-tag). Further amino acid sequences fused with the unmethylated CpG-dinucleotides binding protein may facilitate binding biotin (BCCP-tag), amylose (maltose binding protein-tag) or metal ions. A preferred embodiment is the fusion to a metal ion binding amino acid sequence, particularly to a Hisₙ-tag, n being 3 to 10, preferably 6.

A terminal form of derivatization of the unmethylated CpG-dinucleotides binding protein, i.e. its modification on the N-terminus and/or on the C-terminus for the purpose of a covalent or noncovalent immobilization on base materials, offers the advantage of reducing steric problems in the interaction of the protein with base materials and in the interaction with nucleic acids.

An embodiment of the method in accordance with the invention likewise preferred is that the base material is a metal chelate forming material, in particular a metal chelate forming agarose, preferably a nickel ion binding agarose, particularly preferred sepharose.

According to experimental experience made within the scope of the present invention it is preferred that the unmethylated CpG-dinucleotides binding protein, on its N-terminus, has a His₆-tag and is immobilized on nickel chelate forming sepharose via Ni²⁺ cations.

The present invention in particular makes use of human proteins that bind unmethylated CpG-dinucleotides containing DNA. One of the preferred proteins used for developing a pre-analytic system is CXXC1/CGBP. Of course, it is well known to a skilled person that also all protein isoforms such as, for example, the CXXC1 isoform 1 and the CXXC1 isoform 2 may be used.

As was already mentioned, the CXXC domain is responsible for binding on unmethylated CpG DNA (confer Voo et al., 2000 “Cloning of a mammalian transcriptional activator that binds unmethylated CpG motifs and shares a CXXC Domain with DNA methyltransferase, human trithorax, and methyl-CpG binding Domain protein 1. Mol Cell Biol. Mar; 20(6):2108-21”).

Typically, merely part of the CXXC1 sequence is required, e.g. for the construction of a truncated CXXC1-His-tag fusion protein. The CXXC1 portion includes the CXXC domain and an amino acid sequence of acidic character and was fused, for example, with a Hexa-His-unit and linker sequence that is encoded by the bacterial protein expression vector pET28a(+). The amino acid sequence of the resulting fusion protein is depicted in FIG. 1. This is the so-called Hexa-His-Tag fusion protein CXXC1-P181 (cf. SEQ-ID no. 4 as well as cDNA and isoforms in accordance with SEQ-ID nos. 1 to 3). The charged amino acid residues that are in the acidic region of the sequence facilitate solubility of the protein in physiological buffers.

### BRIEF DESCRIPTION OF THE DRAWINGS

Further advantages and features of the present invention become the description of practical examples making reference to the drawings, wherein:

FIG. 1 provides the sequence of the Hexa-His-Tag Fusion Protein CXXC1-P181;

FIG. 2 illustrates the configuration of the LOOXSTER particle;

FIG. 3 illustrates a chromatographic LOOXSTER binding assay;

FIG. 4 illustrates the interaction of the LOOXSTER column with human genomic DNA and genomic DNA of E. coli;

FIG. 5 illustrates the methylation-sensitive nature of the interaction of the LOOXSTER column with genomic DNA of E. coli;

FIG. 6 illustrates the methylation-sensitive nature of the interaction of the LOOXSTER column with genomic DNA of S. aureaus;

FIG. 7 illustrates the dependence of LOOXSTER-DNA interaction on the CpG-frequency; and

FIG. 8 illustrates the dependence of LOOXSTER-DNA interaction on the G+C content.

### DETAILED DESCRIPTION OF THE DRAWINGS

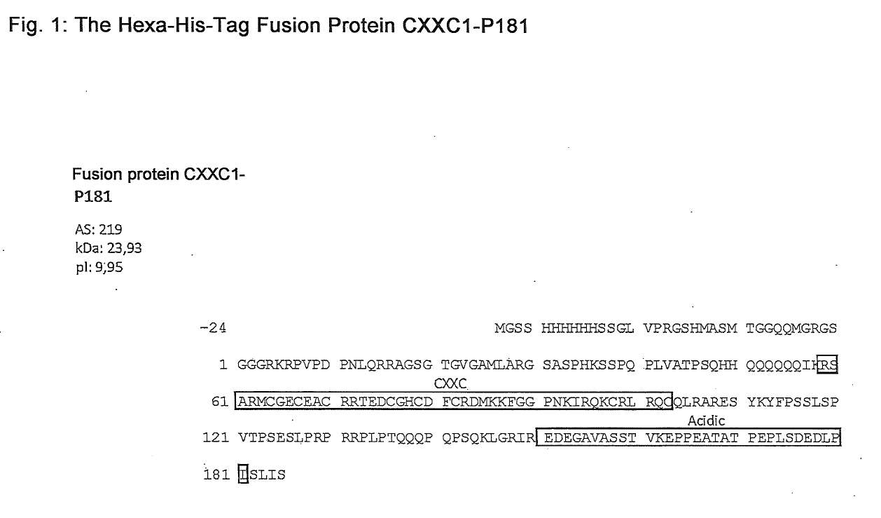

In FIG. 1, functional domains are framed. The fusion protein includes 185 amino acids that originate from the CXXC1 sequence, but is referred to as P181 since the number of amino acids relates to a precursor construct.

In the context with the present invention a general E. coli expression system is used, i.e. the pET28a(+)/E. coli BL21 (DE3) system. With the aid of this system the Hexa-His-Tag fusion protein CXXC1-P181 may be expressed efficiently. The recombinantly produced protein allows for easy solubility in using chaotropic buffers and may be transferred into an active structural status with the aid of so-called On-Column-Refolding.

An active CXXC containing protein or relevant fragments thereof are referred to as “LOOXSTER protein” for the purpose of the present invention (LOOXSTER is a Community Trademark registered under the number GM 004601027 on behalf of Applicant's predecessor SIRS-Lab GmbH, now owned by Analytik Jena AG, Jena, Germany). As an example, the LOOXSTER protein also refers to the CXXC1-P181 protein which, representative for the other proteins binding unmethylated CpG motifs, is used for the detailed description and disclosure of the present invention within the scope of the present embodiments.

The LOOXSTER proteins and in particular the CXXC1-P181 protein may easily be immobilized via their N-terminus His-Tags on different metal-chelating, solid bases. In the context with the present invention a LOOXSTER protein in its immobilized state is referred to as “LOOXSTER particle”.

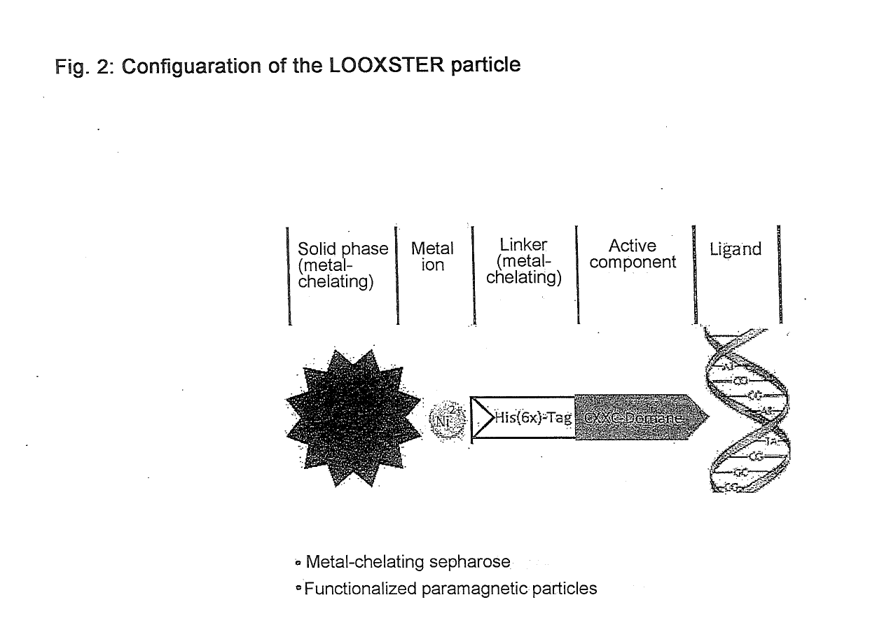

The schematic composition of a LOOXSTER particle is illustrated in FIG. 2. The active component and the chemistry of immobilization allow for only smaller modifications. However, the solid base may add additional chemical and/or physical parameters to the LOOXSTER system. Those parameters include, for example, particle material, particle size, porosity, density or functional groups, spacer, paramagnetism or ferromagnetism.

The fusion protein CXXC1-P181 in particular is immobilized via an N-terminus and/or C-terminus Hexa-His-Tag on the surface of metal-chelating bases. The thus arising particle binding unmethylated CpG motifs may be composed by using bases of different chemical and physical properties. On account of the steric requirements of interaction of the unmethylated CpG motifs binding protein with DNA, terminal immobilization of the protein thereby including the N-terminus and/or the C-terminus of the unmethylated CpG motifs binding protein is preferred. The configuration used in the context of the present invention allows for sufficiently reducing such steric hindrances.

The use of the N-terminus or the C-terminus for immobilization also is suited for minimizing steric problems that may reduce the efficiency of the process.

Depending on the physical properties of the solid base the LOOXSTER particles may be formed as batch or cartridge-/column-based assay systems. A preferred embodiment of the present invention consists in metal-chelated agarose derivates (sepharoses) with a particle size of approximately 90 μm and paramagnetic nickel-chelated beads that have a particle diameter of 2-14 μm. Such a system has turned out to be advantageous for both cartridges and columns, respectively, and batch systems.

The interaction of unmethylated CpG motifs binding proteins with DNA was examined with the aid of affinity chromatography assays. To achieve this, a CXXC1-P181 protein, for example, was immobilized on a nickel sepharose, e.g. Ni-sepharose 6 FF (GE-Healthcare) with a predefined protein density, and the affinity matrix thus produced was used for packing a 1 ml chromatography column. This so-called LOOXSTER column then was connected to a chromatography system (e.g. ÄKTA Purifier; GE-Healthcare). The LOOXSTER chromatography assay consisted of several steps: equilibration, application of the sample, washing out of unbound sample material, elution by way of a linear gradient, maintaining a suitable concentration plateau, reverse gradient and re-equilibration.

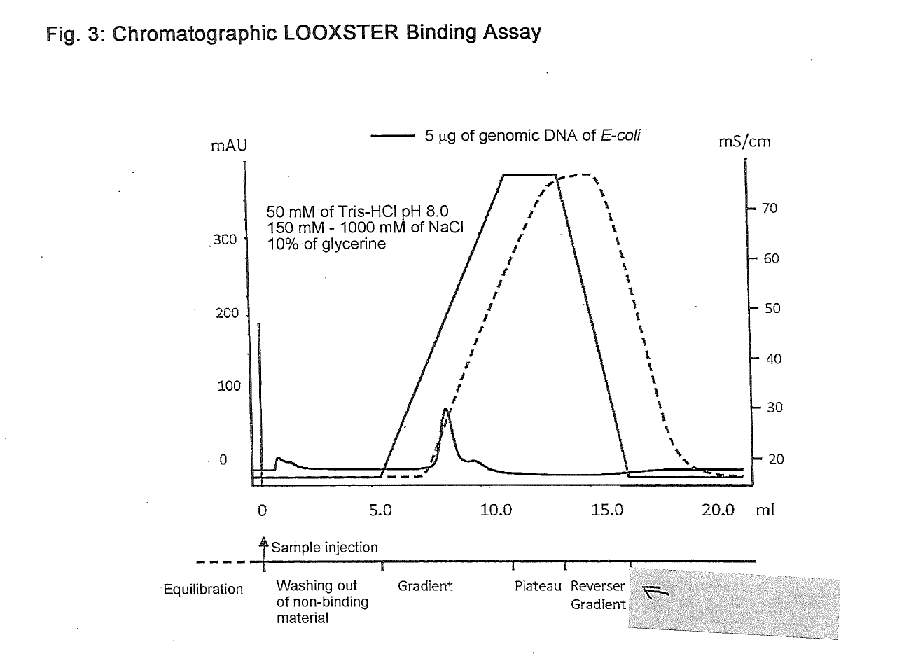

Binding and removing the DNA sample from the LOOXSTER column was performed by changing the NaCl concentration. The UV absorption at 260 nm and the conductivity were measured and recorded. The affinity of the LOOXSTER protein for the DNA sample applied under the given conditions was measured as the conductivity value at the maximum of the absorption peak (confer FIG. 3). The assay was used for testing the interaction of the LOOXSTER system with human genomic DNA and bacterial genomic DNA from different species as well as with genomic DNA of selected species of fungi. The assay was used in particular for determining the influence of different parameters such as the pH value, methylation, frequency of the CpG motifs, etc. on the LOOXSTER-DNA interaction.

FIG. 3 shows a typical chromatogram with the steps of the chromatographic cycle. The UV absorption curve clearly shows that the DNA sample that exclusively contains unmethylated CpG motifs, i.e. the genomic DNA of E. coli, is bound virtually quantitatively to the LOOXSTER column under a low NaCl concentration, and can be eluted from the column by way of a linear gradient with a significant higher NaCl concentration.

Description of the Interaction of the LOOXSTER Protein with DNA

In the present example, an interaction of the LOOXSTER protein with human and bacterial genomic deoxyribonucleic acids was examined, using the assay described above.

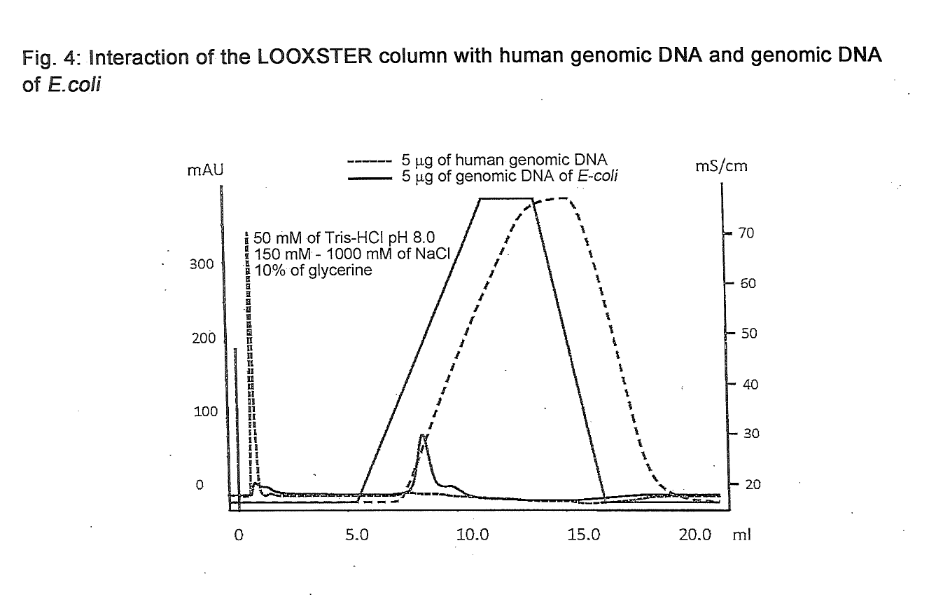

The experiment reveals that under the experimental conditions indicated the LOOXSTER proteins are capable of binding bacterial, exclusively unmethylated CpG-dinucleotides containing DNA, but are not able to significantly interact with human genomic DNA containing methylated CpG-dinucleotides (cf. FIG. 4).

FIG. 4 in particular simulates the binding characteristics for a mixture of human and bacterial DNA. The chromatographic binding studies according to FIG. 4 were performed separately with human and E. coli genomic DNA, respectively (5 μg each), and the data together were plotted in FIG. 4 against the elution volume. Under the present experimental conditions (50 mM of Tris-HCl, pH 8.0, 150-1000 mM of NaCl), the LOOXSTER protein and in particular the CXXC1-p181 virtually quantitatively binds unmethylated genomic DNA of E. coli while there are no significant interactions with human genomic DNA. This is to be discerned in that the human DNA which for the most part includes methylated CpG-dinucleotides, appears as a sharp peak in the column's flow through, whereas the bacterial DNA of E. coli that exclusively includes unmethylated CpG-dinucleotides is eluted from the column only after the application of a specific salt concentration.

It is well known to a skilled person that for the elution of unmethylated CpG-dinucleotides containing DNA, irrespective of its origin, of a LOOXSTER protein, e.g. CXXC1-p181, also other elution agents may be used. Thus, further well water-soluble and non-radioactive alkaline salts and alkaline earth metal salts may be considered as elution agents.

### Methylation Dependency of the LOOXSTER Protein/DNA Interaction

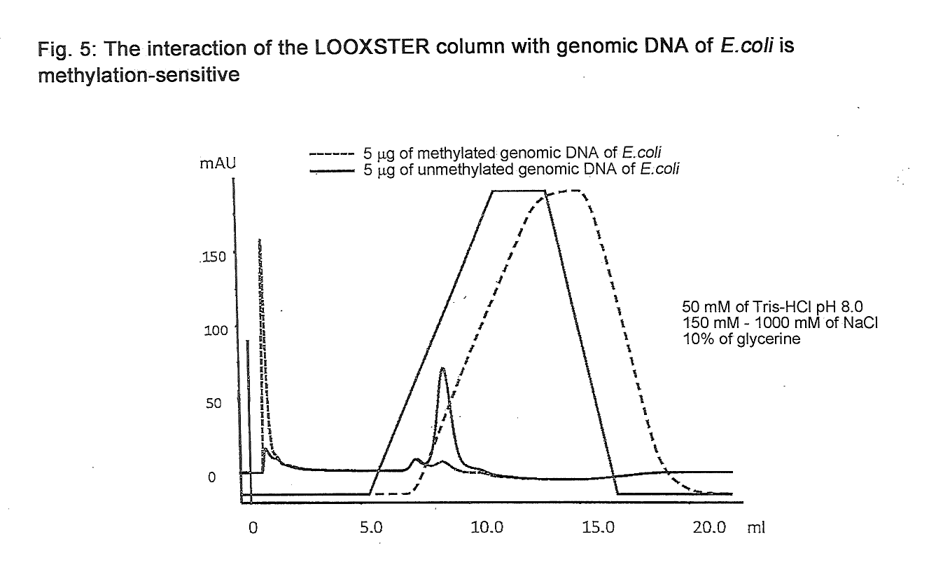

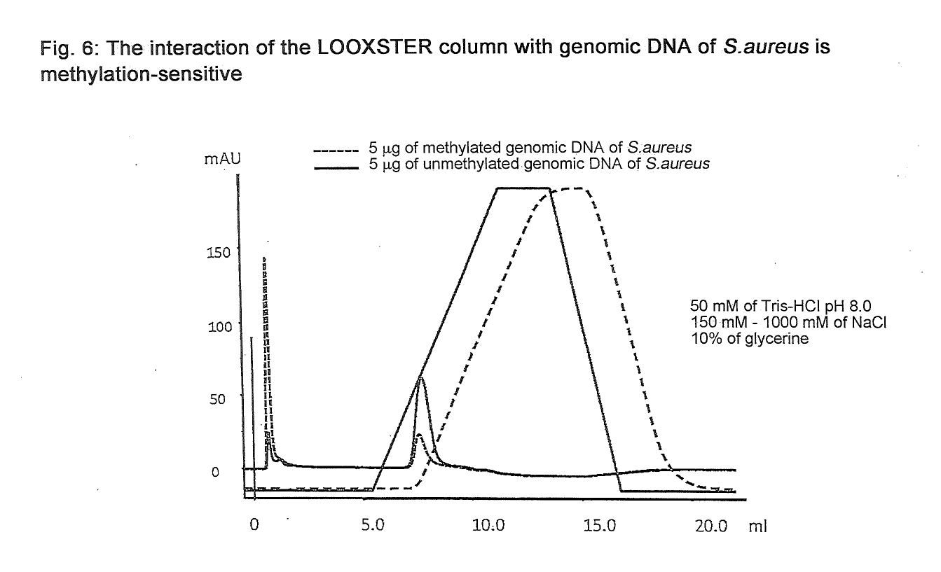

CXXC containing proteins in general and the LOOXSTER proteins used in accordance with the present invention in particular are, as was mentioned before several times, proteins that are capable of binding specifically unmethylated CpG-dinucleotides within the DNA. In order to demonstrate that the interaction of the LOOXSTER proteins in accordance with the invention with DNA depends on its degree of methylation, the following binding studies were performed within the scope of the present application: on a sepharose column with LOOXSTER protein immobilized thereon an affinity chromatography such as, for example, depicted in FIG. 4 was performed. For this, DNA of two different species of bacteria, i.e. E. coli on the one hand, and S. aureus on the other hand, was isolated. The species-specific DNA was then methylated artificially in vitro by way of Sss I methyltransferase and the respective species-specific methylated DNA and the corresponding unchanged native DNA were separately applied to a LOOXSTER affinity column. The chromatograms of the native DNA and the bacterial DNA methylated in vitro for E. coli are shown in FIG. 5. For S. aureus, the results are depicted in FIG. 6. For both species of bacteria it is held that under the given experimental conditions the respective native unmethylated DNA is bound by the LOOXSTER column, while the artificially in vitro methylated DNA preparations of E. coli and S. aureus appear in the column's flow through almost entirely.

### Dependency of the LOOXSTER Protein/DNA Interaction on the Frequency of a CpG Motif within the DNA

A LOOXSTER column that is filled with 1 ml of Ni sepharose on which 1 mg of unmethylated CpG motifs binding protein was immobilized, includes approximately 2.5×10¹⁶ protein molecules. In a LOOXSTER chromatography assay in which 5 μg of E. coli DNA was employed with 9.84×10⁹ genome copies, the stoichiometric proportion of protein and DNA molecules approximately is 2.6×10⁷:1. The binding stoichiometry and the fact that a single DNA molecule typically has numerous CpG binding sites confirms the assumption put forward in the present application that a single DNA molecule is able to interact with proteins binding unmethylated CpG motifs several times.

CpG-dinucleotides are not spread uniformly within the genomic DNA. This fact is made use of by the present invention. It thus is possible that DNA that is rich in unmethylated CpG motifs to a greater degree binds a base with corresponding affinity ligands of unmethylated CpG motifs than DNA that is poor in unmethylated CpG motifs.

However, it is known from the prior art that different CpG-dinucleotides within a single DNA sequence possibly bind CXXC domains not with the same affinity (cf. Lee J H, Voo K S, Skalnik D G (2001): Identification and characterization of the DNA binding domain of CpG-binding protein. J Biol Chem. November 30; 276(48):44669-76. Epub 2001 Sep. 25).

Lee et al. (2001) in particular found out that in the hCGP protein the accompanying nucleotides of the CpG-dinucleotides played a modulating role for the binding affinity. The authors in particular ascertained that those CpG motifs that are accompanied by adenine and thymine, exhibited the highest binding affinity. The resulting motif is the so-called CpG-Consensus-Motif having the sequence: A/C CG A/C.

Both the genomic GC content and the frequency of the CpG-motifs vary strongly between the individual species of microorganisms.

Within the scope of the present invention the binding affinity of the LOOXSTER protein therefore was examined vis-à-vis different genomes of bacteria with different GC contents and CpG motif frequencies.

In so doing, it turned out that it is bijectively possible at least for the species of bacteria rendered in table 2 to correlate the electric conductivity of fractions that were eluted from a column of proteins immobilized on a solid base and binding unmethylated CpG motifs using a specific salt concentration, with the frequency of CpG occurring in a DNA. Thus, by way of suitable calibration, the association of a specific conductivity value, via the CpG frequency and/or the GC content of the DNA molecules to be examined, and the determination of the species of bacteria is possible. Provided that a known species of bacteria is the target organism, a database entry or literature citation with regard to the GC content and/or the CpG frequency may be applied. In case the organism is a microorganism that has not yet been characterized in the databases with regard to its GC content and/or CpG frequency, the data, on the one hand, may be acquired properly by way of sequencing methods well known to a skilled person, determination of the melting temperature, or with the aid of gas chromatography or equilibrium centrifugation, or calibration with other bacteria DNAs may take place that allow for a one-to-one correlation of the target organism with the conductivity as a measure for the concentration of the elution agent, in particular a salt gradient.

TABLE 2
CpG-Motif frequencies and correlation with elution conductivity as
measurement for the concentration of an elution agent for
a number of exemplary species of bacteria

| | 1/CpG- | | | |
|---|---|---|---|---|
| | Motif | GC- | Genome | Elution |
| | frequency | content | size | conductivity |
| Species | [nt] | [%] | [Mbp] | [mS/cm] |
| Pseudomonas aeroginosa | 36.2 | 66.3-66.6 | 6.59 | 31.733 |
| Stenotrophomonas | 36.6 | 66.3 | 4.85 | 31.463 |
| maltophilia | | | | |
| Neisseria meningitidis | 42.8 | 51.5-51.9 | 2.15 | 29.888 |
| Escherichia coli | 55.5 | 50.4-50.9 | 4.75 | 26.742 |
| Proteus mirabilis | 112.1 | 38.9 | 4.06 | 20.63 |
| Acinetobacter baumannii | 121.4 | 38.9-39.4 | 3.98 | 21.272 |
| Streptococcus | 145.0 | 39.5-39.8 | 2.21 | 17.729 |
| pneumoniae | | | | |
| Staphylococcus aureus | 159.7 | 32.8-33.0 | 2.81 | 18.047 |
| Clostridium perfringens | 803.7 | 28.2-28.6 | 3.26 | 18.056 |

Table 2 shows the reciprocal CpG-Consensus motif frequencies, GC contents, genome sizes as well as the measured elution conductivity for a number of exemplary microorganisms.

Here, the numbers of reciprocal CpG motif frequencies in table 2 signify that one CpG motif, respectively, occurs for the nucleotide number [nt] indicated in column 2.

For Pseudomonas aeruginosa this means that 1 CpG motif accounts for 36.2 nucleotides of its genome. For Clostridium perfringens the value amounts to merely 1 CpG motif to 803.7 nucleotides of the C. perfringens genome.

The results obtained within the scope of the present invention clearly prove that the affinity of the LOOXSTER proteins for target DNAs of microorganisms, in form of the affinity chromatography with salt gradient elution as set out above, is determined by the content of CpG motifs, in particular CpG-Consensus motifs that are contained in the DNA of the respective microorganism.

Summing up, it thus is to be ascertained that the CpG frequency can be correlated with the binding affinity of immobilized protein binding unmethylated CpG-dinucleotides (immobilized LOOXSTER protein) and that the eluted fractions can be associated according to ionic strength and concentration of the elution agent, respectively.

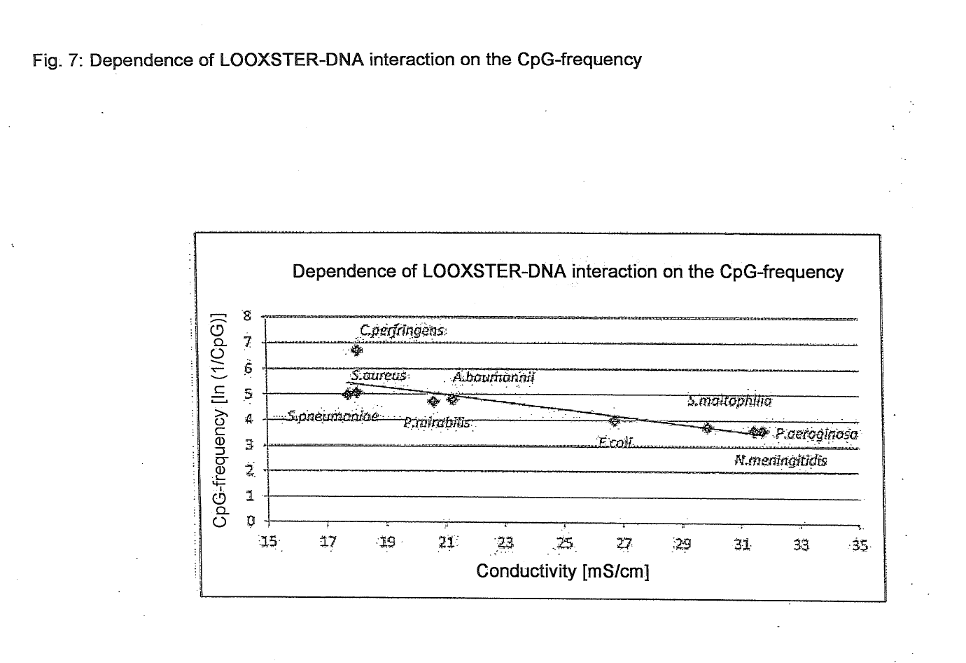

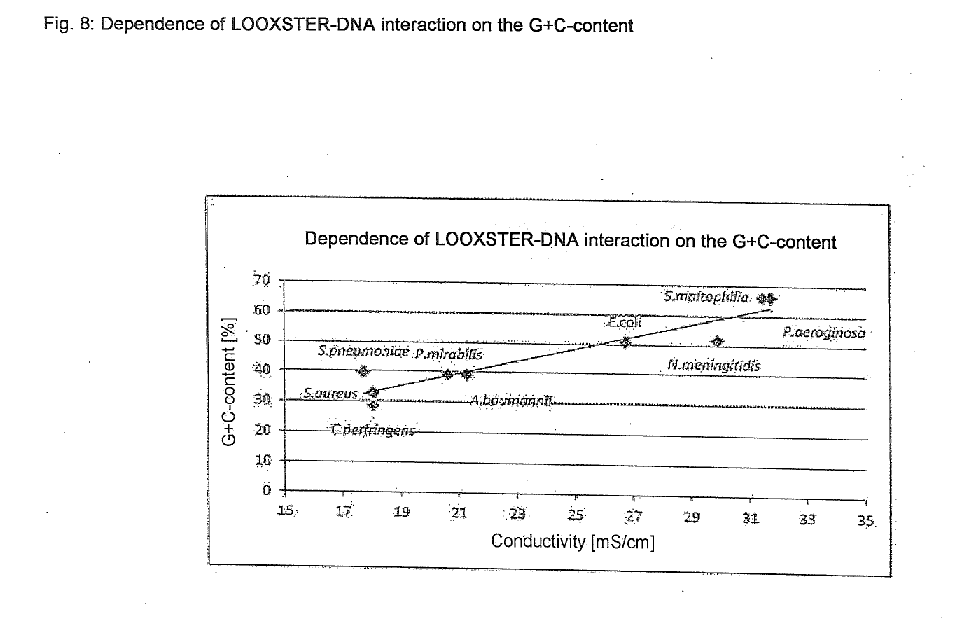

A further proof for this is given in FIG. 7 and FIG. 8. In FIG. 7, the natural logarithm (Ln) of the reciprocal CpG frequency in accordance with table 2 is plotted against the conductivity as measure for ionic strength and concentration, respectively, of each of the eluted fractions as well as the corresponding species, a difference of 10 mM of NaCl corresponding approximately to a conductivity difference of about 1.11 mS/cm. In other words, the abscissa of FIG. 7 indicates the amount of salt concentration (expressed as conductivity) required in order to elute DNA bound to a matrix of immobilized unmethylated CpG motifs binding protein (immobilized LOOXSTER protein).

In FIG. 8, the GC content in accordance with table 2 is plotted against the conductivity as measurement of ionic strength and the concentration, respectively, of each of the eluted fractions as well as the corresponding species, a difference of 10 mM of NaCl corresponding approximately to a conductivity difference of about 1.11 mS/cm. In other words, the abscissa of FIG. 8 indicates the amount of salt concentration (expressed as conductivity) required in order to elute DNA bound to a matrix of immobilized unmethylated CpG motifs binding protein (immobilized LOOXSTER protein).

Under the given experimental conditions (conductivity of the binding buffer: 16.325 mS/cm), human DNA in the indicated example of the LOOXSTER protein does not exhibit an affinity for the LOOXSTER column matrix. Likewise, an influence of the different genome sizes of the bacteria species on a LOOXSTER-DNA-interaction to a large extent could be excluded experimentally.

Consequently, the method in accordance with the invention is suited to separate, determine or enrich different DNA species that are present simultaneously in a sample, on the basis of their different content of unmethylated CpG-dinucleotides and their different degree of methylation.

The method in accordance with the invention may be used for example for identifying pathogens in patients with infections. The method is assigned a particular role in determining pathogens in patients suffering from sepsis.

1-12. (canceled)

13. An in vitro method for separating, determining or enriching different DNA species that are present simultaneously in a sample, on the basis of unmethylated CpG-dinucleotides occurring with varying frequency within each DNA species present, unmethylated CpG-dinucleotides being those that do not comprise a methyl group in position 5 of the cytosine, wherein
the DNA that contains several different DNA species, is isolated from the sample and an aqueous solution of the isolated DNA is produced;
the DNA solution is brought into contact with at least one protein being immobilized on a base material and binding unmethylated CpG-dinucleotides;
unspecifically bound DNA and methylated DNA is eluted; and
the base material is eluted using at least one elution agent that has different concentrations, specific concentration regions of the elution agent used correlating with specific CpG-dinucleotide frequencies in the DNA species present, so that during elution different fractions of DNA species are obtained that have different frequencies of CpG-dinucleotides.

14. The method according to claim 13, characterized in that the different DNA species originate from taxonomically different microorganisms.

15. The method according to claim 14, characterized in that the microorganisms are bacteria and/or fungi that are selected in particular from the group consisting of:
the genera of bacteria:
Pseudomonas; Stenotrophomonas; Neisseriaceae; Echerichia; Proteus; Acinetobacter; Streptococcus; Staphylococcus; Costridium;
the species of bacteria:
Pseudomonas aeroginosa, Stenotrophomonas maltophila, Neisseria meningitidis, Echerichia coli, Proteus mirabilis, Acinetobacter baumannii, Streptococcus pneumoniae, Staphylococcus aureus, Clostridium perfringens;
the fungus genera of Aspergillus and Saccharomyces; and
the fungus species of Aspergillus niger and Saccaromyces cerevisiae.

16. The method according to claim 2, characterized in that the determination of different DNA species is used for identifying microorganisms, in particular genera of bacteria and/or fungi and/or cyanobacteria and/or algae and/or protozoa, and/or species of bacteria and/or cyanobacteria and/or algae and/or fungi and/or protozoa.

17. The method according to claim 16, characterized in that the identification of microorganisms is done on the basis of the detected DNA species in the sample via the following association of frequencies of the CpG-dinucleotide within the detected DNA species, expressed as reciprocal of the frequency (ν) of CpG-dinucleotides, the frequency being the number of nucleotides [nt] in a DNA species that statistically accounts for one CpG-dinucleotide:

| Species of microorganism | 1/ν CpG-dinucleotide [nt] |
|---|---|
| Pseudomonas aeruginosa | 36.0-36.4, preferably 36.2 |
| Stenotrophomonas maltophilia | 36.5-36.8, preferably 36.6 |
| Neisseria meningitidis | 42.4-43.2, preferably 42.8 |
| Echerichia coli | 55.1-55.9, preferably 55.5 |
| Proteus mirabilis | 111.5-112.6, preferably 112.1 |
| Acinetobacter baumannii | 120.9-121.9, preferably 121.4 |
| Streptococcus pneumoniae | 144.5-145.5, preferably 145.0 |
| Staphylococcus aureus | 159.2-160.2, preferably 159.7 |
| Clostridium perfringens | 801.7-805.7, preferably 803.7 |

18. The method according to claim 13, characterized in that as measurement of the frequency of a CpG-dinucleotide in a DNA species the content of the nucleobases guanine and cytosine (GC-content) is used, expressed in % as
$$[ \frac{mGuanine + mCytosine}{mAdenine + mThymine + mGuanine + mCytosine} ] \times 100$$
m referring to the mass of the respective nucleobase.

19. The method according to claim 13, characterized in that the base is eluted using a salt solution, in particular a salt gradient, preferably an (NH₄)₂CO₃-gradient or an NaCl gradient having an NaCl concentration of 0.01 M to 1.0 M, particularly 0.1 M to 0.8 M, preferably 0.2 M to 0.7 M, wherein specific concentration regions correspond to a specific CpG frequency and/or a specific GC-content, so that individual DNA fractions are obtained that have different CpG frequencies and/or different GC-contents; and
comparing the salt concentrations of the individual eluted DNA fractions with the salt concentration of eluted DNA with a known CpG frequency and/or known GC-content of known microorganisms in order to identify an unknown microorganism on the basis of its CpG frequency and/or GC-content in the respective DNA fraction.

20. The method according to claim 19, characterized in that the salt concentration of the individual DNA fractions is determined via a measurement of the electrical conductivity.

21. The method according to claim 13, characterized in that the unmethylated CpG-dinucleotides binding protein is selected from the group consisting of: proteins containing a CXXC domain; protein fragments comprising at least one CXXC domain, particularly human non-methyl-CpG binding protein CXXC1/CGBP as well as its isoforms 1 and 2 and fragments thereof that include the CXXC domain; fusion proteins including the CXXC domain.

22. The method according to claim 21, characterized in that the unmethylated CpG-dinucleotide binding protein on its N-terminus and/or C-terminus has a metal ion binding linker, in particular a Hisₙ-tag, n being 3 to 10, preferably 6.

23. The method according to claim 13, characterized in that the base material is a metal chelate forming material, in particular a metal chelate forming agarose, preferably a nickel ion binding agarose, particularly preferred sepharose.

24. The method according to claim 23, characterized in that the unmethylated CpG-dinucleotides binding protein, on its N- and/or C terminus, has a His₆-tag and is immobilized on nickel chelate forming sepharose via Ni²⁺ cations.


SEQUENCE LISTING (4 sequences)

SEQ ID NO: 1 - DNA, 2976 bp, Homo sapiens
  source 1..2976: mRNA NM_001101654.1
ggaaagagtggtggcaggtgaagtcggagacgacagaggaactggtttcctccgccccgcaaggcacacagcctgccgacgccccattaatacatgtggaaggggaaagagactgaatggaggaatgaatacaacttgatccaggtcgtgcttcggaagcggtcactttacctgtgaacctctctgcctgacaaacgggcaatgtacggaatcaaccaccaagatggcggcgcccgtgaagaatccgcaattaggtcgccgtcatatgtcgcctaggaacgtacggaattcgacccacgtacggaatcggattccaagatgacggcatctatgaggaagtcacgcagtaggtgcagccatgttgcctgtacgtcgaggccgtacaagcagccgccgtacggactctactgacaaggtggcggcgccctcgggaaagccacattagagcgcggccatgttcccggcgaacatatggattcggccaccatacggatacgataagcaagatggcggcgcctgaggggtcttgggggctctaggccggccacctactggtttgcagcggagacgacgcatggggcctgcgcaataggagtacgctgcctgggaggcgtgactagaagcggaagtagttgtgggcgcctttgcaaccgcctgggacgccgccgagtggtctgtgcaggttcgcgggtcgctggcgggggtcgtgagggagtgcgccgggagcggagatatggagggagatggttcagacccagagcctccagatgccggggaggacagcaagtccgagaatggggagaatgcgcccatctactgcatctgccgcaaaccggacatcaactgcttcatgatcgggtgtgacaactgcaatgagtggttccatggggactgcatccggatcactgagaagatggccaaggccatccgggagtggtactgtcgggagtgcagagagaaagaccccaagctagagattcgctatcggcacaagaagtcacgggagcgggatggcaatgagcgggacagcagtgagccccgggatgagggtggagggcgcaagaggcctgtccctgatccagacctgcagcgccgggcagggtcagggacaggggttggggccatgcttgctcggggctctgcttcgccccacaaatcctctccgcagcccttggtggccacacccagccagcatcaccagcagcagcagcagcagatcaaacggtcagcccgcatgtgtggtgagtgtgaggcatgtcggcgcactgaggactgtggtcactgtgatttctgtcgggacatgaagaagttcgggggccccaacaagatccggcagaagtgccggctgcgccagtgccagctgcgggcccgggaatcgtacaagtacttcccttcctcgctctcaccagtgacgccctcagagtccctgccaaggccccgccggccactgcccacccaacagcagccacagccatcacagaagttagggcgcatccgtgaagatgagggggcagtggcgtcatcaacagtcaaggagcctcctgaggctacagccacacctgagccactctcagatgaggacctacctctggatcctgacctgtatcaggacttctgtgcaggggcctttgatgaccatggcctgccctggatgagcgacacagaagagtccccattcctggaccccgcgctgcggaagagggcagtgaaagtgaagcatgtgaagcgtcgggagaagaagtctgagaagaaggtgatggagaggaaggaggagcgatacaagcggcatcggcagaagcagaagcacaaggataaatggaaacacccagagagggctgatgccaaggaccctgcgtcactgccccagtgcctggggcccggctgtgtgcgccccgcccagcccagctccaagtattgctcagatgactgtggcatgaagctggcagccaaccgcatctacgagatcctcccccagcgcatccagcagtggcagcagagcccttgcattgctgaagagcacggcaagaagctgctcgaacgcattcgccgagagcagcagagtgcccgcactcgccttcaggaaatggaacgccgattccatgagcttgaggccatcattctacgtgccaagcagcaggctgtgcgcgaggatgaggagagcaacgagggtgacagtgatgacacagacctgcagatcttctgtgtttcctgtgggcaccccatcaacccacgtgttgccttgcgccacatggagcgctgctacgccaagtatgagagccagacgtcctttgggtccatgtaccccacacgcattgaaggggccacacgactcttctgtgatgtgtataatcctcagagcaaaacatactgtaagcggctccaggtgctgtgccccgagcactcacgggaccccaaagtgccagctgacgaggtatgcgggtgcccccttgtacgtgatgtctttgagctcacgggtgacttctgccgcctgcccaagcgccagtgcaatcgccattactgctgggagaagctgcggcgtgcggaagtggacttggagcgcgtgcgtgtgtggtacaagctggacgagctgtttgagcaggagcgcaatgtgcgcacagccatgacaaaccgcgcgggattgctggccctgatgctgcaccagacgatccagcacgatcccctcactaccgacctgcgctccagtgccgaccgctgagcctcctggcccggaccccttacaccctgcattccagatgggggagccgcccggtgcccgtgtgtccgttcctccactcatctgtttctccggttctccctgtgcccatccaccggttgaccgcccatctgcctttatcagagggactgtccccgtcgacatgttcagtgcctggtggggctgcggagtccactcatccttgcctcctctccctgggttttgttaataaaattttgaagaaaccaaggaaaaaaaaaaaa

SEQ ID NO: 2 - DNA, 2964 bp, Homo sapiens
  source 1..2964: mRNA NM_014593.3
ggaaagagtggtggcaggtgaagtcggagacgacagaggaactggtttcctccgccccgcaaggcacacagcctgccgacgccccattaatacatgtggaaggggaaagagactgaatggaggaatgaatacaacttgatccaggtcgtgcttcggaagcggtcactttacctgtgaacctctctgcctgacaaacgggcaatgtacggaatcaaccaccaagatggcggcgcccgtgaagaatccgcaattaggtcgccgtcatatgtcgcctaggaacgtacggaattcgacccacgtacggaatcggattccaagatgacggcatctatgaggaagtcacgcagtaggtgcagccatgttgcctgtacgtcgaggccgtacaagcagccgccgtacggactctactgacaaggtggcggcgccctcgggaaagccacattagagcgcggccatgttcccggcgaacatatggattcggccaccatacggatacgataagcaagatggcggcgcctgaggggtcttgggggctctaggccggccacctactggtttgcagcggagacgacgcatggggcctgcgcaataggagtacgctgcctgggaggcgtgactagaagcggaagtagttgtgggcgcctttgcaaccgcctgggacgccgccgagtggtctgtgcaggttcgcgggtcgctggcgggggtcgtgagggagtgcgccgggagcggagatatggagggagatggttcagacccagagcctccagatgccggggaggacagcaagtccgagaatggggagaatgcgcccatctactgcatctgccgcaaaccggacatcaactgcttcatgatcgggtgtgacaactgcaatgagtggttccatggggactgcatccggatcactgagaagatggccaaggccatccgggagtggtactgtcgggagtgcagagagaaagaccccaagctagagattcgctatcggcacaagaagtcacgggagcgggatggcaatgagcgggacagcagtgagccccgggatgagggtggagggcgcaagaggcctgtccctgatccagacctgcagcgccgggcagggtcagggacaggggttggggccatgcttgctcggggctctgcttcgccccacaaatcctctccgcagcccttggtggccacacccagccagcatcaccagcagcagcagcagcagatcaaacggtcagcccgcatgtgtggtgagtgtgaggcatgtcggcgcactgaggactgtggtcactgtgatttctgtcgggacatgaagaagttcgggggccccaacaagatccggcagaagtgccggctgcgccagtgccagctgcgggcccgggaatcgtacaagtacttcccttcctcgctctcaccagtgacgccctcagagtccctgccaaggccccgccggccactgcccacccaacagcagccacagccatcacagaagttagggcgcatccgtgaagatgagggggcagtggcgtcatcaacagtcaaggagcctcctgaggctacagccacacctgagccactctcagatgaggacctacctctggatcctgacctgtatcaggacttctgtgcaggggcctttgatgaccatggcctgccctggatgagcgacacagaagagtccccattcctggaccccgcgctgcggaagagggcagtgaaagtgaagcatgtgaagcgtcgggagaagaagtctgagaagaagaaggaggagcgatacaagcggcatcggcagaagcagaagcacaaggataaatggaaacacccagagagggctgatgccaaggaccctgcgtcactgccccagtgcctggggcccggctgtgtgcgccccgcccagcccagctccaagtattgctcagatgactgtggcatgaagctggcagccaaccgcatctacgagatcctcccccagcgcatccagcagtggcagcagagcccttgcattgctgaagagcacggcaagaagctgctcgaacgcattcgccgagagcagcagagtgcccgcactcgccttcaggaaatggaacgccgattccatgagcttgaggccatcattctacgtgccaagcagcaggctgtgcgcgaggatgaggagagcaacgagggtgacagtgatgacacagacctgcagatcttctgtgtttcctgtgggcaccccatcaacccacgtgttgccttgcgccacatggagcgctgctacgccaagtatgagagccagacgtcctttgggtccatgtaccccacacgcattgaaggggccacacgactcttctgtgatgtgtataatcctcagagcaaaacatactgtaagcggctccaggtgctgtgccccgagcactcacgggaccccaaagtgccagctgacgaggtatgcgggtgcccccttgtacgtgatgtctttgagctcacgggtgacttctgccgcctgcccaagcgccagtgcaatcgccattactgctgggagaagctgcggcgtgcggaagtggacttggagcgcgtgcgtgtgtggtacaagctggacgagctgtttgagcaggagcgcaatgtgcgcacagccatgacaaaccgcgcgggattgctggccctgatgctgcaccagacgatccagcacgatcccctcactaccgacctgcgctccagtgccgaccgctgagcctcctggcccggaccccttacaccctgcattccagatgggggagccgcccggtgcccgtgtgtccgttcctccactcatctgtttctccggttctccctgtgcccatccaccggttgaccgcccatctgcctttatcagagggactgtccccgtcgacatgttcagtgcctggtggggctgcggagtccactcatccttgcctcctctccctgggttttgttaataaaattttgaagaaaccaaggaaaaaaaaaaaa

SEQ ID NO: 3 - DNA, 570 bp, Homo sapiens
  source 1..570: other DNA CXXC1-Insert (BamHI/EcoRI) into pET28a
ggatccggtggagggcgcaagaggcctgtccctgatccaaacctgcagcgccgggcagggtcagggacaggggttggggccatgcttgctcggggctctgcttcgccccacaaatcctctccgcagcccttggtggccacacccagccagcatcaccagcagcagcagcagcagatcaaacggtcagcccgcatgtgtggtgagtgtgaggcatgtcggcgcactgaggactgtggtcactgtgatttctgtcgggacatgaagaagttcgggggccccaacaagatccggcagaagtgccggctgcgccagtgccagctgcgggcccgggaatcgtacaagtacttcccttcctcgctctcaccagtgacgccctcagagtccctgccaaggccccgccggccactgcccacccaacagcagccacagccatcacagaagttagggcgcatccgtgaagatgagggggcagtggcgtcatcaacagtcaaggagcctcctgaggctacagccacacctgagccactctcagatgaggacctacctctaagcttaattagctgagaattc

SEQ ID NO: 4 - protein, 219 aa, Homo sapiens
MGSSHHHHHHSSGLVPRGSHMASMTGGQQMGRGSGGGRKRPVPDPNLQRRAGSGTGVGAMLARGSASPHKSSPQPLVATPSQHHQQQQQQIKRSARMCGECEACRRTEDCGHCDFCRDMKKFGGPNKIRQKCRLRQCQLRARESYKYFPSSLSPVTPSESLPRPRRPLPTQQQPQPSQKLGRIREDEGAVASSTVKEPPEATATPEPLSDEDLPLSLIS